(19) United States
(12) Patent Application Publication
(10) Pub. No.: US 2017/0260574 A1
(43) Pub. Date: Sep. 14, 2017

(54) Rapid Gene Sensors from Carbon Nanotube-DNA Systems

(71) Applicants: Juan Noveron, El Paso, TX (US); Hugo Alarcon, El Paso, TX (US)

(72) Inventors: Juan Noveron, El Paso, TX (US); Hugo Alarcon, El Paso, TX (US)

(21) Appl. No.: 15/407,909

(22) Filed: Jan. 17, 2017

Related U.S. Application Data
(60) Provisional application No. 62/279,172, filed on Jan. 15, 2016.

(51) Int. Cl. C12Q 1/68; G01N 21/65; G01N 21/77

(52) U.S. Cl. CPC C12Q 1/6816; C12Q 1/6806; C12Q 1/6848; G01N 21/7703; G01N 21/65

(57) ABSTRACT

Methods, devices, and/or systems for providing carbon nanotube material that interacts with nucleotides to form CNT-nucleotide nanostructures wherein the CNT-nucleotide nanostructures form detectable network structures upon reactions with nucleic acids having targeted sequences.

### PRIORITY PARAGRAPH

This Application claims priority to U.S. Provisional Patent application Ser. No. 62/279,172 filed Jan. 15, 2016, which is incorporated herein by reference in its entirety.

### STATEMENT REGARDING FEDERALLY FUNDED RESEARCH

This invention was made with government support under 0748913 and 301-496-1776 awarded by the National Science Foundation and the National Institutes of Health, respectively. The government has certain rights in the invention.

### BACKGROUND

Embodiments described herein are related to the field of nucleic acid sequence detection and to the uses thereof, especially in medicine and health care and in particular medical diagnostics.

It is contemplated that devices that detect the conserved genes of viruses or other pathogens in real-time will develop into technologies that can be applied in the field and speed up critical medical interventions of a wide variety of infectious diseases (Zhai et al., 1997). Currently, genetic based diagnosis is based on placing fluorescent tags on nucleic acid sequences followed with optical spectroscopy techniques (Ferguson et al., 1996) or involve non-real-time intricate protocols (Taylor et al., 1996); however, these methods are time-consuming and are not cost-effective for rapid practical applications.

Recently the ability of DNA to bind carbon nanotubes (CNTs) has been established (Zheng et al., Nat. Mater. 2003; Zheng et al., Science 2003). DNA has been shown to form π-stacking complexes resulting in helical wrapping of the CNT surface. Thus, the use of carbon-nanotube-based applications in biotechnology has been contemplated. However, the use of carbon-nanotube-based real time nucleic acid detection or nucleic acid detection capabilities in the field has not yet previously been disclosed.

### SUMMARY

Embodiments of the invention are directed to compositions, devices, and methods of using and making carbon nanotube (CNT) materials that interact with nucleotides, such as DNA, to form CNT-nucleotide nanostructures. These nanostructures may be configured to form network structures, such as hydrogels or aggregates, upon reactions with nucleic acid target sequences. These network structures are detectable structures that may be used as gene-sensing technology.

As further disclosed herein, the technology can be applied in many fields. In particular the technology can be applied to the medical field. The technology can be applied to gene-sensing technologies. Gene-sensing technologies can include, but are not limited to medical diagnostics of pathogen infections.

Certain embodiments are directed to carbon nanotube probes comprising a functionalized carbon nanotube coupled to one or more nucleic acid probes that bind a target, wherein two or more carbon nanotube probes associate in the presence of the target forming a carbon nanotube network comprising a plurality of carbon nanotube probes and a plurality of targets. In certain aspects the nucleic acid probe is non-covalently bound to the carbon nanotube. In other aspects the nucleic acid probe is covalently bound to the carbon nanotube. The nucleic acid probe can be a DNA, RNA, or DNA and RNA probe. In certain aspects the nucleic acid probe is at least 15, 20, 25, 30, 35, 40, 45, 50 or more nucleotides in length. In a further aspect the nucleic acid probe is at least 50 nucleotides in length. The carbon nanotube can be a single walled or multi-walled carbon nanotube. In certain aspects the carbon nanotubes can be 5 nm to 5 μm in length. In still a further aspect the carbon nanotubes can have an outer diameter of 1 nm to 10 nm. The carbon nanotube can have a length to diameter ratio of 5 to 1,000,000. In certain aspects the carbon nanotube is functionalized with at least one group defined as:

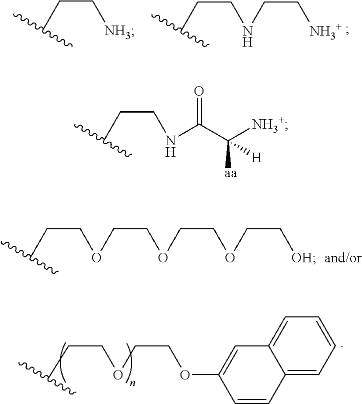

Other embodiments are directed to carbon nanotube networks comprising a plurality of carbon nanotube probes as described herein and a plurality of targets, wherein association of the carbon nanotube probes induced by the target forms a carbon nanotube network. In certain aspects the target is a nucleic acid. In a further aspect the target nucleic acid is a single stranded nucleic acid. The nucleic acid can be DNA, RNA, or a combination thereof. The carbon nanotube network can be a detectable carbon nanotube network. In certain aspects the carbon nanotube network forms a hydrogel or an aggregate. The carbon nanotube network can be detected, for example, by eye, bright field optical microscopy, resonance raman spectroscopy, differential pulse voltammetry, and/or dynamic light scattering.

Certain embodiments are directed to methods for detecting a single-stranded or double-stranded nucleic acid having a target sequence comprising the steps of: (a) contacting a sample suspected of containing said nucleic acid having a target sequence with a carbon nanotube probe as described herein, wherein the carbon nanotube probe is configured to form a network structure upon contact with a nucleic acid having a target sequence; (b) detecting the presence of the nucleic acid having a target sequence by detecting a carbon nanotube probe network structure that is formed in the presence of a target nucleic acid.

Other embodiments are directed to processes for making a carbon nanotube probe composition comprising: (a) functionalizing a carbon nanotube with a functional group that is capable of binding at least one nucleotide and/or nucleic acid; (b) contacting the functionalized carbon nanotube with at least one nucleotide and/or nucleic acid wherein the at least one nucleotide is configured to bind a nucleic acid having a target sequence; (c) sonicating the solution containing the functionalized carbon nanotube and at least one nucleotide and/or nucleic acid.

The samples suspected of having a target sequence used in the present invention include any samples. Preferably, the samples are biological samples. The biological samples may be plant, animal, human, fungus, bacterium, virus, or combinations thereof. Samples of a mammal or human can be derived from a particular body fluid, tissue, or organ. A biological sample may include any cell, tissue, or fluid from a biological source, including food.

Throughout this application, the term “hybridizing” refers to the formation of a double-stranded nucleic acid from complementary single stranded nucleic acids. A complementary single stranded nucleic acid may be perfectly matched or substantially matched with some mismatches to its complement. Complementarity for hybridization may depend on hybridization conditions, such as temperature.

Throughout this application, the term “target nucleic acid”, “target nucleic acid sequence”, “target sequence”, or “nucleic acid having a target sequence” refers to a nucleic acid sequence of interest for detection. A target nucleic acid to which the detection method of the present invention can be applied is not particularly limited as long as the nucleic acid has a sequence that can hybridize with a probe sequence. Such a nucleic acid that can be utilized may be any of DNA and RNA and may be any of single-strand and double-strand nucleic acids. The base sequence of such a nucleic acid that can be used includes at least a portion of a gene or genome sequence to be detected. The presence or absence of the nucleic acid including such a sequence can be detected. The nucleic acid is not limited by origin. Specifically, any of natural nucleic acids (e.g., animal-, plant-, microorganism- and virus-derived nucleic acids) and artificially synthesized nucleic acids (e.g., chemically synthesized nucleic acids and nucleic acids synthesized in a gene engineering manner) can be utilized as a target nucleic acid.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Sequence-dependent aggregation (network structure) of single-wall carbon nanotubes-single stranded DNA (SWCNT-ssDNA) nanostructures.

FIG. 2(a) Chemical structure of functionalized SWCNTs 1-5. (b) Bright field optical microscopy of 1. (c) Resonance Raman of 1 reveals CNT-specific absorbance. (d) Dynamic light scattering of 1 in aqueous media indicate homogeneous dispersion in solution.

FIG. 3 Sequence-dependent aggregation of SWCNT(1).

FIG. 4 Real-time gene sensing technology of infectious diseases.

### DESCRIPTION

Carbon nanotubes (CNTs) are allotropes of carbon with a cylindrical nanostructure. CNTs are members of the fullerene structural family. Their name is derived from their long, hollow structure with the walls formed by one-atom-thick sheets of carbon, called graphene. These sheets are rolled at specific and discrete (“chiral”) angles, and the combination of the rolling angle and radius decides the nanotube properties; for example, whether the individual nanotube shell is a metal or semiconductor. Nanotubes are categorized as single-walled nanotubes (SWNTs) and multi-walled nanotubes (MWNTs). Nanotubes have been constructed with length-to-diameter ratio of up to 132,000,000:1, significantly larger than for any other material. These cylindrical carbon molecules have unusual properties, which are valuable for nanotechnology, electronics, optics and other fields of materials science and technology.

Embodiments are directed to compositions, devices, and methods of using and making of CNT material that interacts with nucleotides or nucleic acids such as DNA to form CNT-nucleotide nanostructures that are capable of forming network structures, such as hydrogels or arrogates, upon binding or aggregation with nucleic acid(s) having a target sequence. In some instances, this technology works by means of designed nanostructures that polymerize or aggregate upon binding with a target nucleic acid. In some instances, the polymerization of the composition is sequence specific to the nucleic acid having a target sequence.

In some aspects, the network structure may be detected by direct or indirect visualization. In one aspect the network structure is detected by bright field optical microscopy, resonance raman, dynamic light scattering, and/or differential pulse voltammetry, etc. In a further aspect detection can be determined by a change in viscosity or gelling of a probe solution.

In some aspects, the CNT-nucleotide material forms a network structure when in contact with nM or lower concentrations of nucleic acids with target sequences. In one instance, the CNT-nucleotide may detect in real time the presence of nucleic acids with target sequences.

Compositions and methods described herein can be applied in many fields, including medical diagnostics and microbe detection. In some instances, compositions and methods described herein can be applied in real-time nucleic acids sensors and in nanofluidics based rapid diagnostic technologies.

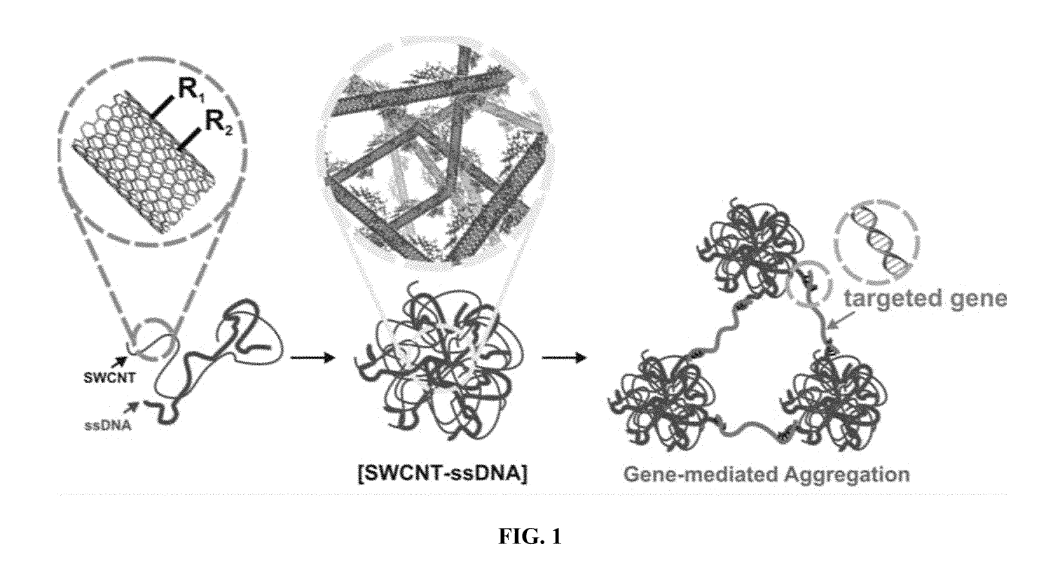

FIG. 1 provides a non-limiting example of this approach. Single-wall carbon nanotubes (SWCNTs) and DNA materials can be used in real-time detection of specific nucleic acid sequences. In one instance, this new technology works by means of designed nanostructures that polymerize or aggregate upon reaction with targeted nucleic acids as shown in FIG. 1. Aggregation of the nanostructures induced by a nucleic acid having a specific sequence may then be detected by bright field optical microscopy, resonance raman, dynamic light scattering, and/or differential pulse voltammetry, etc.

Embodiments are directed to carbon nanotube probe compositions and related methods for detecting a variety of pathogens or potential pathogens (e.g., NIAID Category A, B, and C priority pathogens). In particular aspects of the invention the compositions and methods of the invention may be used to detect a biological weapon or opportunistic microbe.

There are numerous microbes that are considered pathogenic or potentially pathogenic under certain conditions (i.e., opportunistic pathogens/microbes). Bacterial microbes that can be detected using compositions and methods described herein include, but are not limited to various species of the Bacillus, Yersinia, Franscisella, Streptococcus, Staphylococcus, Pseudomonas, Mycobacterium, Burkholderia genus of bacteria. Particular species of bacteria that can be detected include, but is not limited to Bacillus anthracis, Yersinia pestis, Francisella tularensis, Streptococcus pnemoniae, Staphylococcus aureas, Pseudomonas aeruginosa, Burkholderia cepacia, Corynebacterium diphtherias, Clostridia spp, Shigella spp., and Mycobacterium avium.

There are numerous viruses and viral strains that can be detected using the compositions or methods described herein. Viruses can be placed in one of the seven following groups: Group I: double-stranded DNA viruses, Group II: single-stranded DNA viruses, Group III: double-stranded RNA viruses, Group IV: positive-sense single-stranded RNA viruses, Group V: negative-sense single-stranded RNA viruses, Group VI: reverse transcribing Diploid single-stranded RNA viruses, Group VII: reverse transcribing Circular double-stranded DNA viruses. Viruses include the family Adenoviridae, Arenaviridae, Caliciviridae, Coronaviridae, Filoviridae, Flaviviridae, Hepadnaviridae, Herpesviridae (Alphaherpesvirinae, Betaherpesvirinae, Gammaherpesvirinae), Nidovirales, Papillomaviridae, Paramyxoviridae (Paramyxovirinae, Pneumovirinae), Parvoviridae (Parvovirinae, Picornaviridae), Poxyiridae (Chordopoxyirinae), Reoviridae, Retroviridae (Orthoretrovirinae), and/or Togaviridae. These virus include, but are not limited to various strains of influenza, such as avian flu (e.g., H5N1). Particular virus from which a subject may be protected include, but is not limited to Cytomegalovirus, Respiratory syncytial virus and the like. Examples of pathogenic virus that can be detected include, but are not limited to Influenza A, H5N1, Marburg, Ebola, Dengue, Severe acute respiratory syndrome coronavirus, Yellow fever virus, Human respiratory syncytial virus, Vaccinia virus and the like.

There are numerous fungal species that are considered pathogenic or potentially pathogenic under certain conditions that can be detected using the compositions and methods described herein. Fungi include, but are not limited to Aspergillus fumigatus, Candida albicans, Cryptococcus neoformans, Histoplasma capsulatum, Coccidioides immitis, Pneumocystis carinii, and Blastomyces dermatitidis.

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation and Characterization of Functionalized CNTs

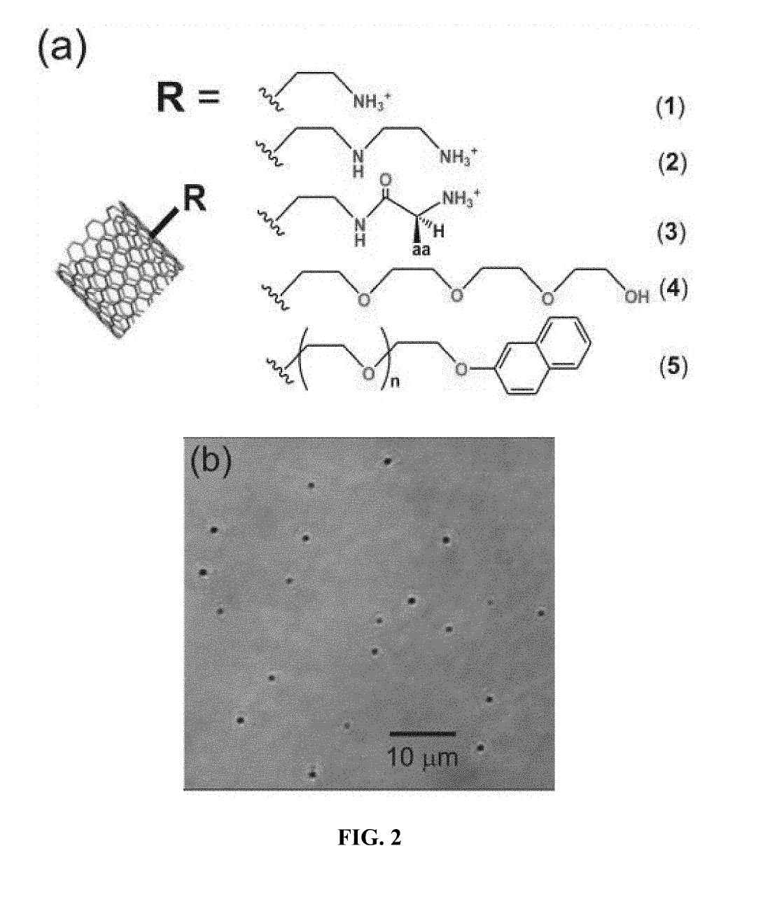

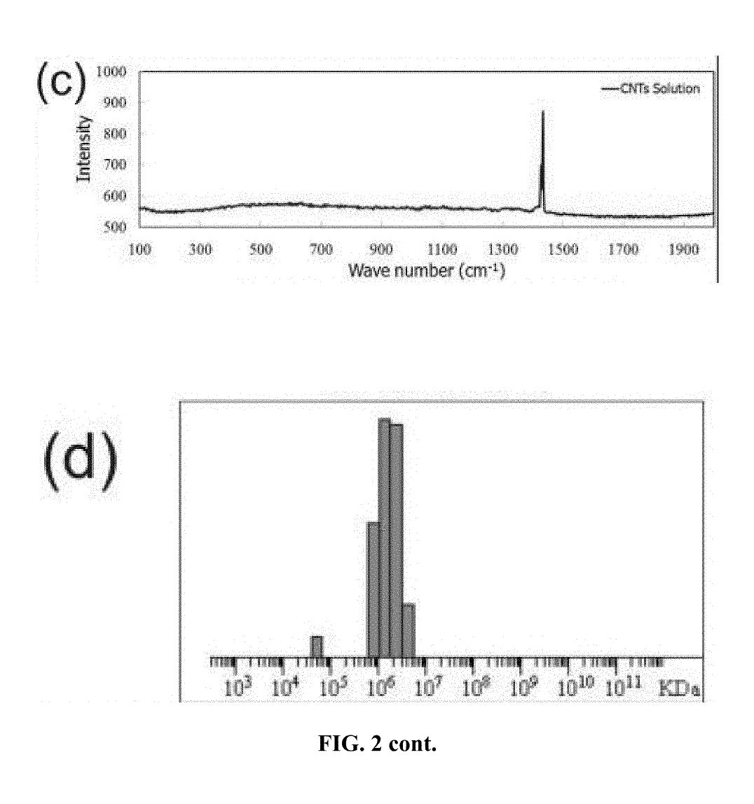

Functionalized CNTs—Several functionalized single-wall carbon nanotubes (SWCNTs) (1-5) have been prepared and characterized, FIG. 2(a), following established synthetic protocols (Mickelson et al., 1999; Bahr et al., 2001; Holzinger et al., 2001; Georgakilas et al., 2002). These functionalized SWCNTs (1-5) were shown to be capable of making stable suspensions in in aqueous media while increasing their affinity to bind DNA.

Characterization of CNTs—Optical microscopy, resonance raman, and dynamic light scattering were used to determine the stability in water and dispersion in water of the functionalized SWCNTs. It was determined that the functionalized SWCNTs form stable homogenous dispersions in water suitable for further reactions with DNA. FIG. 2(b)-FIG. 2(d) shows data results of SWCNT(1) characterization, which is representative of SWCNT's (2)-(5).

### EXAMPLE 2

### Preparation and Characterization of CNT-Nucleotides

CNT-nucleotides—Functionalized SWCNT(1) was reacted with single stranded DNA composed of C₁₂A₁₂, where C=cytosine and A=adenine, by sonication in the presence of the single-stranded DNA according to the methods described in Zheng et al., Nat. Mater. 2003 and Zheng et al., Science 2003. The product of the reaction was a SWCNT bound by the C₁₂A₁₂ nucleotide (1-DNA(C₁₂A₁₂)).

Characterization of CNT-nucleotides—The network structure of 1-DNA(C₁₂A₁₂) was determined by dynamic light scattering. 1-DNA(C₁₂A₁₂) forms stable bundles of discrete sizes similar to those illustrated in FIG. 1 (top, middle section).

### EXAMPLE 3

### Sensing Nucleic Acids Using CNT-Nucleotide

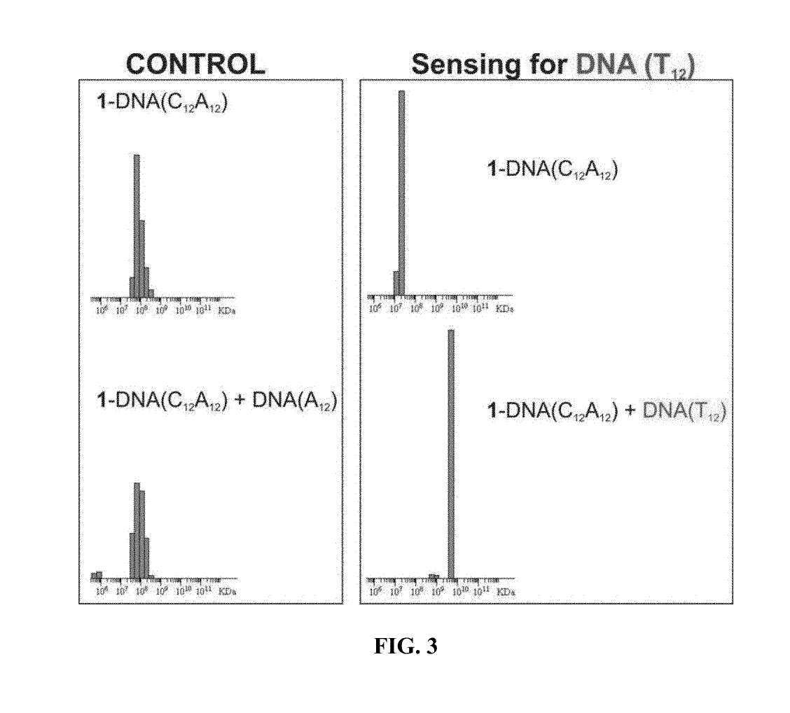

The network structure of a CNT-nucleotide was determined with and without the presence of a target nucleic acid. The network structure of the following solutions were determined by dynamic light scattering: 1-DNA(C₁₂A₁₂) solution of Example 2; 1-DNA(C₁₂A₁₂) exposed to a target single stranded DNA composed of T24 (DNA(T₁₂)), where T=thymine; and 1-DNA(C₁₂A₁₂) exposed to a non-target control single strand DNA composed of A₁₂ (DNA((A₁₂)), where A=adenine. It was determined that aggregation is only triggered by the targeted DNA sequence (FIG. 3). It was also determined that aggregation occurred when the CNT-nucleotide was contacted with target nucleic acid at nM concentrations.

### EXAMPLE 4

### Sensing Nucleic Acids S Using SWCNT-ssDNA

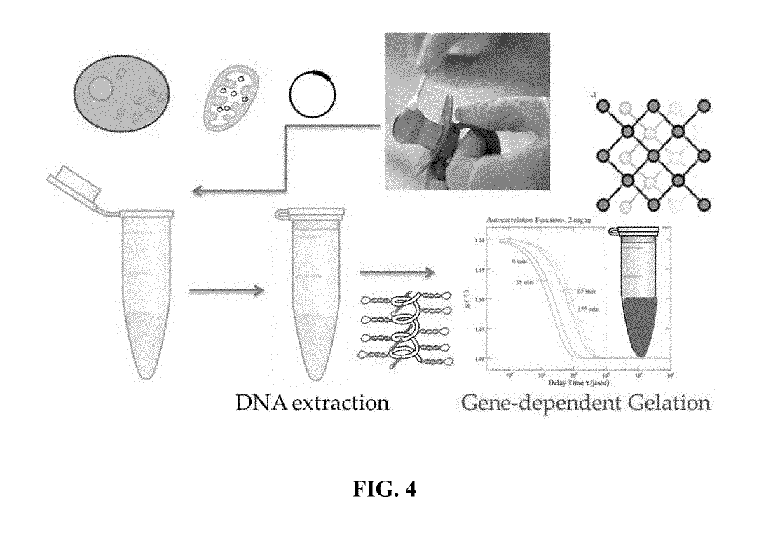

SWCNT-nucleotide technology as described herein can be used for the real-time sensing of conserved single-stranded RNA from influenza virus, for example. The SWCNT-nucleotide technology can be directed to highly conserved regions of virus as the target sequence. An example of application of this technology is outlined in FIG. 4.

The technology described herein can be used as real-time gene sensors of nucleic acids such as those of infectious diseases. It is further contemplated that this technology can be used in the context of nanofluidics for rapid diagnosis in medical applications.

### REFERENCES

Bahr et al., J. Am. Chem. Soc. 2001, 123, 6536.
Ferguson et al., Nat. Biotechnol.,1996, 14, 1681.
Georgakilas et al., Chem. Commun. 2002, 3050.
Holzinger et al., Angew. Chem., Int. Ed. 2001, 40, 4002.
Mickelson et al., J. Phys. Chem. B 1999, 103, 4318.
Taylor and Schultz, Handbook of Chemical and Biological Sensors. Institute of Physics Publishing, Bristol, UK, 1996.
Zhai et al., Biotechnol. Adv.,1997, 15, 43.
Zheng et al., Nat. Mater. 2003, 2, 338.
Zheng et al., Science 2003, 302, 1545.

1. A carbon nanotube probe comprising a functionalized carbon nanotube coupled to one or more nucleic acid probes that bind a target, wherein two or more carbon nanotube probes associate in the presence of the target forming a carbon nanotube network comprising a plurality of carbon nanotube probes and a plurality of targets.

2. The composition of claim 1, wherein the nucleic acid probe is non-covalently bound to the carbon nanotube.

3. The composition of claim 1, wherein the nucleic acid probe is covalently bound to the carbon nanotube.

4. The composition of claim 1, wherein the nucleic acid probe is DNA, RNA, or DNA and RNA.

5. The composition of claim 1, wherein the nucleic acid probe is at least 20 nucleotides.

6. The composition of claim 1, wherein the nucleic acid probe is at least 50 nucleotides.

7. The composition of claim 1, wherein the carbon nanotube is a single walled carbon nanotube.

8. The composition of claim 1, wherein the carbon nanotubes are 5 nm to 5 μm in length.

9. The composition of claim 1, wherein the carbon nanotubes have an outer diameter of 1 nm to 10 nm.

10. The composition of claim 1, wherein the carbon nanotube has a length to diameter ratio of 5 to 1,000,000.

11. The composition of claim 8, wherein the carbon nanotube is functionalized with at least one group defined as:

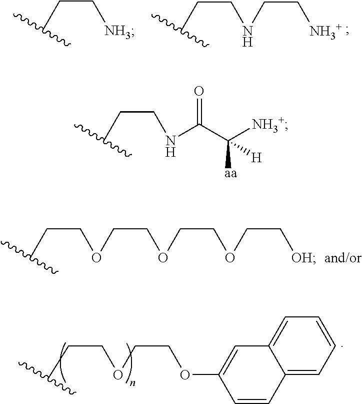

12. A carbon nanotube network comprising a plurality of carbon nanotubes coupled to one or more nucleic acid probes and a plurality of targets, wherein association of the carbon nanotube probes induced by the target forms a carbon nanotube network.

13. The network of claim 12, wherein the target is a nucleic acid.

14. The network of claim 13, wherein the nucleic acid is a single stranded nucleic acid.

15. The network of claim 13, wherein the nucleic acid is DNA or RNA.

16. The network of claim 12, wherein the carbon nanotube network is a detectable carbon nanotube network.

17. The composition of claim 12, wherein the carbon nanotube network is a hydrogel or an aggregate.

18. The composition of claim 12, wherein the carbon nanotube network is detectable by bright field optical microscopy, resonance raman spectroscopy, differential pulse voltammetry, and/or dynamic light scattering.

19. A method for detecting a single-stranded or double-stranded nucleic acid having a target sequence comprising the steps of:
(a) contacting a sample suspected of containing said nucleic acid having a target sequence with a carbon nanotube probe of claim 1, wherein the carbon nanotube probe is configured to form a network structure upon contact with a nucleic acid having a target sequence;
(b) detecting the presence of the nucleic acid having a target sequence by detecting a carbon nanotube probe network structure that is formed in the presence of a target nucleic acid.

20. A process for making a carbon nanotube probe composition comprising:
(a) functionalizing a carbon nanotube with a functional group that is capable of binding at least one nucleotide and/or nucleic acid;
(b) contacting the functionalized carbon nanotube with at least one nucleotide and/or nucleic acid wherein the at least one nucleotide is configured to bind a nucleic acid having a target sequence;
(c) sonicating the solution containing the functionalized carbon nanotube and at least one nucleotide and/or nucleic acid.
